(19) United States
(12) Patent Application Publication
(10) Pub. No.: US 2006/0293594 A1
(43) Pub. Date: Dec. 28, 2006

(54) Device for carrying out intravascular examinations

(75) Inventor: Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/473,400

(22) Filed: Jun. 23, 2006

(30) Foreign Application Priority Data
Jun. 24, 2005 (DE) 10 2005 029 476.6

(51) Int. Cl. A61B 5/05

(52) U.S. Cl. 600/425

(57) ABSTRACT

The invention relates to a device for carrying out intravascular examinations, with which image data generated by an intravascular imaging device and electrocardiography signals measured by an electrocardiography device are displayed by means of a device for simultaneously displaying in subareas of a monitor.

### CROSS REFERENCE TO RELATED APPLICATIONS

This application claims priority of German application No. 10 2005 029 476.6 filed Jun. 24, 2005, which is incorporated by reference herein in its entirety.

### FIELD OF THE INVENTION

The invention relates to a device for carrying out intravascular examinations.

### BACKGROUND OF THE INVENTION

According to the prior art, a number of methods are known for carrying out intravascular examinations. A distinction is made here between invasive methods and non-invasive methods. With invasive methods, catheters are used, at the tips of which one or a number of sensors are provided to detect signals, from which image data suitable for producing an image can be generated. Optical coherence tomography (OCT) known from WO 97/32182 A1 for instance is an invasive intravascular method. Furthermore, to produce intravascular images, ultrasound methods such as those described in EP 0 885 594 B1 for instance or magnetic resonance methods (NMR) such as those described in U.S. Pat. No. 6,377,048 B1 for instance are known.

X-ray methods which are generally known according to the prior art are particular examples of non-invasive methods.

Particularly for producing images according to the OCT methods, it is necessary temporarily to seal the vessel to be displayed using a balloon and to displace the blood located therein by means of a rinsing fluid. If the vessel to be examined is located in the region of the cardiac muscle, a temporary interruption of the blood circulation can result in a disturbance of cardiac activity. To be able to identify a disturbance of this kind immediately, cardiac activity is monitored at the same time by means of an electrocardiography device (ECG) according to the prior art. The signals generated by the ECG device are usually displayed on a first monitor connected to the ECG device. The images produced with the intravascular imaging device are displayed according to the prior art on a second monitor, which forms part of the intravascular imaging device. The first and second monitors are usually arranged at a certain distance from one another at the treatment site. To observe both monitors simultaneously, it is disadvantageously necessary for the treating doctor to continually switch their field of vision from one monitor to the other. This requires greater concentration and thus causes fatigue unnecessarily quickly.

### BACKGROUND OF THE INVENTION

The object of the invention is to eliminate the disadvantages according to the prior art. In particular, a device for carrying out intravascular examinations is to be specified, which enables the relevant images and signals to be observed in an improved manner.

This object and expedient embodiments of the invention are achieved by the features of the claims.

According to the invention, provision is made for a device for carrying out intravascular examinations, with
an intravascular imaging device for generating image data,
an electrocardiography (ECG) device for measuring ECG signals and
a monitor
being linked for data transmission to a device for simultaneously displaying an image generated from the image data in a first predetermined subarea of the monitor and a graph showing the currently measured ECG signals in a second predetermined subarea of the monitor.

By simultaneously displaying the image and the graph in predetermined subareas of a single monitor, as proposed in accordance with the invention, it is no longer necessary for the treating doctor to switch their field of vision continually, as required according to the prior art to observe the relevant signals and images. This allows fatigue-free operation. The doctor can concentrate better on the examination, the movement of a catheter for instance. The proposed simultaneous display of the image and graph on a single monitor in accordance with the invention also enables a more rapid response in the event of a complication. The proposed device allows intravascular examinations to be carried out in a safer manner for patients.

According to an advantageous embodiment of the invention, the intravascular imaging device comprises at least one of the following devices:
optical coherence tomography (OCT) device,
ultrasound tomography device,
magnetic resonance (NMR) tomography device,
x-ray device.

The intravascular imaging device can also feature combinations of the said devices. This allows especially precise and largely artifact-free images to be produced. In this case, a suitable image processing device can additionally be provided, with which the image data supplied by the devices can be registered and/or superimposed to generate a common image.

According to a further embodiment, at least one further predetermined subarea can be generated with the device to display a further image and/or a further graph simultaneously. The further image can be a three-dimensional display of a segment of the examined vessel segment for instance. Furthermore, data showing specific life functions of the patient can be displayed in a further graph. The further graph can thus be a curve representation or even a simple display of figures, such as blood pressure values or other data for instance.

The device expediently comprises a computer with a program for generating the subareas and for displaying the image and graph at the same time. Programs of this type are generally known according to the prior art. They typically allow the size and the arrangement of the predetermined subareas and/or windows to be selected.

According to a further embodiment, the signals, a derivation of the signals or an electrocardiogram generated from the signals can be displayed as a graph. In this case, the device thus comprises suitable means for generating the derivation of the signals or for generating an electrocardiogram from the signals.

According to a further embodiment, provision is made for a cable link and/or a transmitter/receiver for wireless data transmission to be provided for data transmission. The latter can comprise a Bluetooth or an IR interface or suchlike.

Furthermore, a storage device for storing images and/or ECG signals in a time-correlated manner can be provided. This allows the data obtained during the examination to be documented and/or archived. A subsequent more advanced evaluation of data is further possible. The storage device can form a part of the device for the simultaneous display.

According to a further particularly advantageous embodiment of the invention, the monitor comprises a subarea, in which a content of at least one subarea can be changed by touching predetermined function fields. The monitor can thus be a so-called “touchscreen”. By touching the function fields provided in the subarea, further images of further intravascular devices can be displayed for instance and derivations of signals or suitably calculated graphs can be selected and displayed. The additional integration of function fields and their operability on the monitor further facilitates the operation of the device. Instead of the proposed “touchscreen”, functions can naturally also be controlled by means of a mouse or suchlike.

### BRIEF DESCRIPTION OF THE DRAWINGS

An exemplary embodiment of the invention is described in more detail below with reference to the drawings, in which;

FIG. 1 shows a schematic diagram of the essential components of the device,

FIG. 2 shows a first monitor and

FIG. 3 shows a second monitor

### DETAILED DESCRIPTION OF THE INVENTION

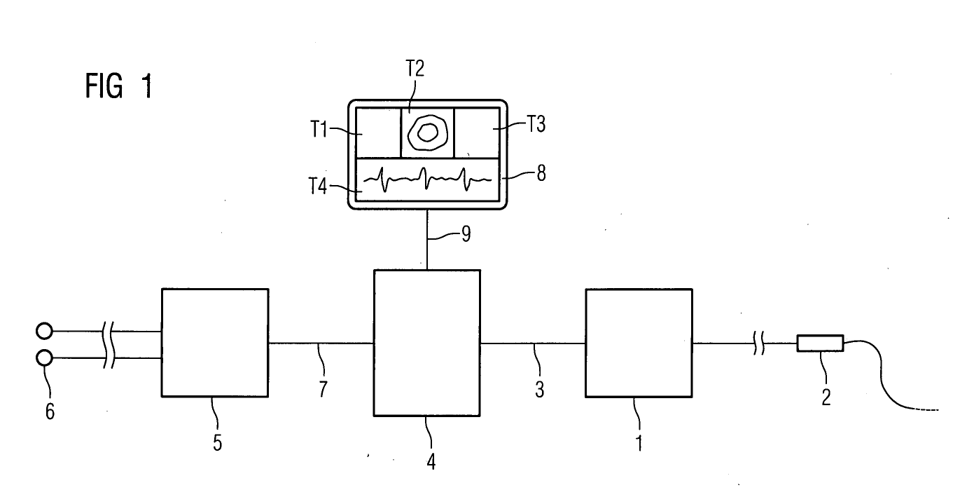

In FIG. 1, an intravascular imaging device 1, for instance an OCT device, is provided with a catheter 2. The intravascular imaging device 1 is connected to a computer 4 via a first cable 3. An ECG device 5 is provided with ECG sensors 6 in order to detect ECG signals. It is likewise connected to the computer 4 via a second cable 7. A monitor identified with the reference character 8 is connected to the computer 4 via a third cable 9. The monitor 8 is divided up into a number of subareas T1, . . . , T4.

The function of the device is as follows:

Signals suitable for generating image data are generated by means of a sensor device provided on the catheter 2 (not shown here). The signals are converted into image data in the intravascular imaging device 1 and are transmitted to the computer 4 via the first cable 3. The image data is processed into an image in the computer 4 and is displayed in one of the subareas T1, . . . , T4 on the monitor 8.

Signals supplied by the ECG sensors 6 by means of the ECG device 5 are likewise converted into graphic data suitable for displaying and are transferred to the computer 4 via the second cable 7. The graphic data is converted in the computer 4 into a format suitable for displaying in one of the subareas T1, . . . T4, and is subsequently displayed on the monitor 8 in one of the subareas T1, . . . T4.

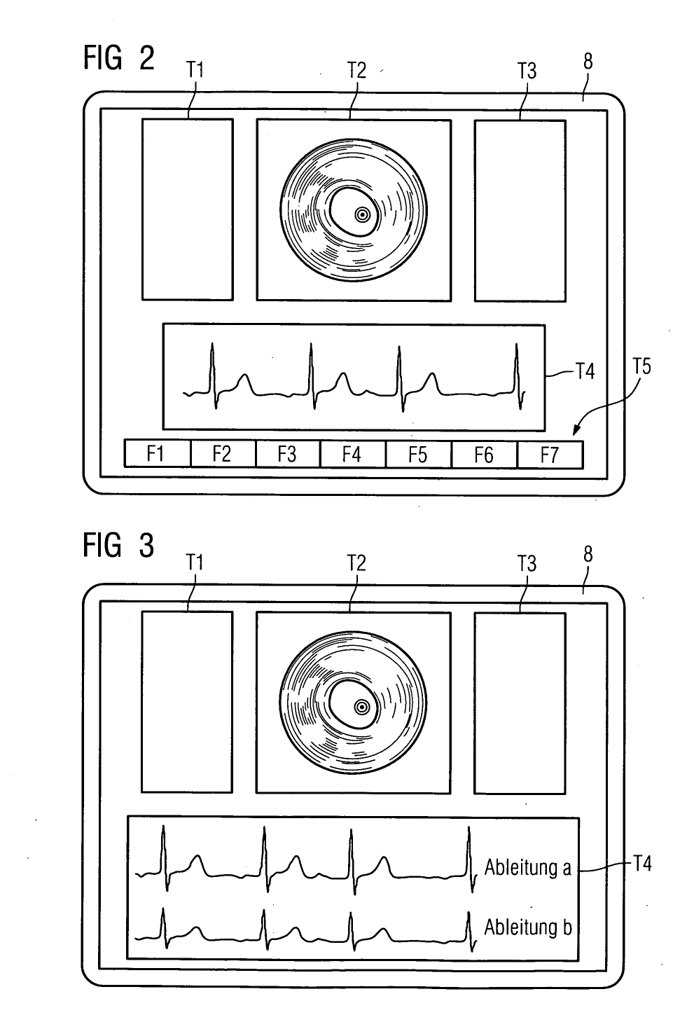

FIG. 2 shows a first monitor 8. The first monitor 8 is divided up into five subareas T1, . . . , T5. The subareas T1, T3 here do not feature any image content. Optionally they can be additionally provided with image content according to a predetermined program. Image content of this type can be generated for instance by further intravascular imaging devices, for example ultrasound devices or NMR devices. One or a number of subareas T1, . . . T5 can also be used to display general information, for instance the current status of the balloon or the sealing time of a vessel effected by the balloon.

In the second subarea indicated here with T2, a two-dimensional image generated by means of an OCT device is displayed. In a fourth subarea T4, the current signals supplied by the ECG device 5 are displayed continuously. A fifth subarea T5 comprises function fields F1, . . . , F7. By touching the function fields F1, . . . F7, the treating doctor can thus trigger predetermined functions, for instance changing the content shown in the subareas T1, . . . T5 or displaying said content in a different display. The monitor 8 shown in FIG. 2 is thus a touch-sensitive monitor and/or touchscreen suitable for triggering functions.

FIG. 3 shows signals derived from ECG signals in the fourth subarea T4. Two derivations are shown here in the fourth subarea T4. In other words, a number of derived signals and/or images can thus also be shown in a subarea T1, . . . , T4.

1-8. (canceled)

9. A device for carrying out an intravascular examination, comprising:
an intravascular imaging device for generating an image data;
an electrocardiography device for measuring an electrocardiography signal;
a monitor; and
a processing device linked with the intravascular imaging device, the electrocardiography device, and the monitor for simultaneously displaying an image generated from the image data in a first predetermined subarea of the monitor and a graph showing the measured electrocardiography signal in a second predetermined subarea of the monitor.

10. The device as claimed in claim 9, wherein the intravascular imaging device comprises a device selected from the group consisting of: optical coherence tomography device, ultrasound tomography device, magnetic resonance tomography device, and x-ray device.

11. The device as claimed in claim 9, wherein a further predetermined subarea is generated with the processing device to display a further image simultaneously.

12. The device as claimed in claim 9, wherein a further predetermined subarea is generated with the processing device to display a further graph simultaneously.

13. The device as claimed in claim 12, wherein a signal, a derivation of the signal or an electrocardiogram generated from the signal is displayed as the graph.

14. The device as claimed in claim 9, wherein the processing device comprises a computer having a computing program for generating predetermined subareas and for simultaneously displaying the image and the graph.

15. The device as claimed in claim 14, wherein a cable link is provided for data transmission.

16. The device as claimed in claim 14, wherein a transmitter and a receiver are provided for wireless data transmission.

17. The device as claimed in claim 16, wherein the transmitter and the receiver comprises a Bluetooth or an IR interface.

18. The device as claimed in claim 9, wherein a storage device is provided for storing the image data.

19. The device as claimed in claim 9, wherein a storage device is provided for storing the electrocardiography signal.

20. The device as claimed in claim 9, wherein the monitor comprises a subarea in which a content of the subarea is changed by touching a predetermined function field.

21. A method for carrying out an intravascular examination, comprising:
generating an image data with an intravascular imaging device;
measuring an electrocardiography signal with an electrocardiography device;
linking the intravascular imaging device, the electrocardiography device, and a monitor to a processing device; and
simultaneously displaying an image generated from the image data in a first predetermined subarea of the monitor and a graph showing the measured electrocardiography signal in a second predetermined subarea of the monitor with the processing device.

22. The method as claimed in claim 21, wherein a further predetermined subarea is generated with the processing device to display a further image simultaneously.

23. The method as claimed in claim 21, wherein a further predetermined subarea is generated with the processing device to display a further graph simultaneously.
